(19) United States
(12) Patent Application Publication
(10) Pub. No.: US 2017/0105645 A1
(43) Pub. Date: Apr. 20, 2017

(54) ELECTROENCEPHALOGRAPHY WITH APPLICATION OF TRANSCRANIAL MAGNETIC STIMULATION

(71) Applicant: YEDA RESEARCH AND DEVELOPMENT CO. LTD., Rehovot (IL)

(72) Inventors: Elisha MOSES, Rehovot (IL); Jodie Naim FEIL, Rehovot (IL); Dominik FRECHE, Rehovot (IL); Nava LEVIT-BINNUN, Rehovot (IL)

(21) Appl. No.: 15/292,147

(22) Filed: Oct. 13, 2016

Related U.S. Application Data
(60) Provisional application No. 62/243,230, filed on Oct. 19, 2015.

(51) Int. Cl. A61B 5/0478; A61N 2/00; A61B 5/0408

(52) U.S. Cl. CPC A61B 5/0478; A61B 5/04085; A61N 2/004

(57) ABSTRACT

A system includes an array of electroencephalography (EEG) electrodes for placement on the head of a subject and a transcranial magnetic stimulation (TMS) device. A controller is configured to operate the array of EEG electrodes to acquire a plurality of EEG signals concurrently with operation of the TMS device to apply a TMS pulse to the head of the subject. The controller is further configured to select an initial set of EEG signals from the EEG signals and to fit an initial decreasing rational function of time to a TMS artifact component of each EEG signal of the initial set. The TMS artifact component of each remaining EEG signal is fit to a linear combination of the initial decreasing rational functions. Each EEG signal is corrected by removing from the EEG signal the function that is fit to the TMS artifact component of the EEG signal.

### CROSS REFERENCE TO RELATED APPLICATIONS

The present invention claims the priority benefit of U.S. provisional patent application No. 62/243,230 filed on Oct. 19, 2015, which is incorporated in its entirety herein by reference.

### FIELD OF THE INVENTION

The present invention relates to electroencephalography. More particularly, the present invention relates to electroencephalography in the presence of transcranial magnetic stimulation.

### BACKGROUND OF THE INVENTION

Transcranial magnetic stimulation (TMS) is often used to stimulate the brain. For example, TMS may activate a brain function (e.g., to reproduce the effects on the brain of performance of a particular activity, in a manner that may be more reliable or reproducible than actual performance of the activity). TMS may be applied in order to achieve the effects of electroconvulsive therapy (ECT) or to induce plasticity in the brain.

The combined use of TMS with electroencephalography (EEG) has become a well-established method in neuroscience for mapping brain activity. TMS and EEG may be used for functional cortical mapping and may potentially serve as a biomarker.

The concomitant application of TMS and EEG may be challenging. The application of a TMS electromagnetic pulse may significantly affect the measured EEG signal. Technical advances have led to improved amplifiers that allow continuous EEG recording during pulse application without amplifier saturation. However, the TMS pulse may lead to large artifacts in the EEG recording. These artifacts may be orders of magnitude larger than the contribution of physiological brain activity to the EEG signal, and may persist for up to hundreds of milliseconds. As a result, it may be difficult or impossible to directly obtain physiological information from the artifactual EEG without further experimental precautions or additional algorithmic treatment.

### SUMMARY OF THE INVENTION

There is thus provided, in accordance with an embodiment of the present invention, a system including: an array of electroencephalography (EEG) electrodes for placement on the head of a subject; a transcranial magnetic stimulation (TMS) device; and a controller configured to operate the array of EEG electrodes to acquire a plurality of EEG signals concurrently with operation of the TMS device to apply a TMS pulse to the head of the subject, the controller further configured to: select an initial set of EEG signals from the plurality of EEG signals; fit an initial decreasing rational function of time to a TMS artifact component of each EEG signal of the initial set; fit the TMS artifact component of each remaining EEG signal of the plurality of EEG signals to a linear combination of the initial decreasing rational functions; and correct each EEG signal of the plurality of EEG signals by removing from the each EEG signal the function that is fit to the TMS artifact component of the each EEG signal.

Furthermore, in accordance with an embodiment of the present invention, the controller is configured to identify the artifact component of each EEG signal by identifying an artifact approximation period in which each EEG signal is dominated by its artifact component.

Furthermore, in accordance with an embodiment of the present invention, the artifact approximation period is empirically determined.

Furthermore, in accordance with an embodiment of the present invention, the controller is configured to select the initial set by selecting a maximum EEG signal and a minimum EEG signal.

Furthermore, in accordance with an embodiment of the present invention, the initial decreasing rational function is expressible as a quadratic polynomial in time divided by a quartic polynomial in time.

Furthermore, in accordance with an embodiment of the present invention, the controller is configured to fit the linear combination to the each remaining EEG signal by determining values of linear combination coefficients.

Furthermore, in accordance with an embodiment of the present invention, the controller is configured to fit the linear combination to the each remaining EEG signal by iteratively alternating between determining the values of linear combination coefficients and determining values of term coefficients.

Furthermore, in accordance with an embodiment of the present invention, the controller is configured to correct each EEG signal of the plurality of EEG signals by subtracting from the each EEG signal the function that is fit to the TMS artifact component of the each EEG signal.

There is further provided, in accordance with an embodiment of the present invention, a controller of a system for acquiring EEG signals concurrently with application of a TMS pulse, the controller configured to: operate an array of EEG electrodes to acquire a plurality of EEG signals concurrently with operation of a TMS device to apply a TMS pulse to the head of the subject; select an initial set of EEG signals from the plurality of EEG signals; fit an initial decreasing rational function to a TMS artifact component of each EEG signal of the initial set; fit the TMS artifact component of each remaining EEG signal of the plurality of EEG signals to a linear combination of the initial decreasing rational functions; and remove the modeled TMS artifact component from each EEG signal of the plurality of EEG signals.

Furthermore, in accordance with an embodiment of the present invention, the controller is configured to identify the artifact component of each EEG signal by identifying an artifact approximation period in which each EEG signal is dominated by its artifact component.

Furthermore, in accordance with an embodiment of the present invention, the artifact approximation period is empirically determined.

Furthermore, in accordance with an embodiment of the present invention, the controller is configured to select the initial set by selecting a maximum EEG signal and a minimum EEG signal.

Furthermore, in accordance with an embodiment of the present invention, the initial decreasing rational function is expressible as a quadratic polynomial in time divided by a quartic polynomial in time.

Furthermore, in accordance with an embodiment of the present invention, the controller is configured to fit the linear combination to the each remaining EEG signal by determining values of linear combination coefficients.

Furthermore, in accordance with an embodiment of the present invention, the controller is configured to fit the linear combination to the each remaining EEG signal by iteratively alternating between determining the values of linear combination coefficients and determining values of term coefficients.

Furthermore, in accordance with an embodiment of the present invention, the controller is configured to correct each EEG signal of the plurality of EEG signals by subtracting from the each EEG signal the function that is fit to the TMS artifact component of the each EEG signal.

There is further provided, in accordance with an embodiment of the present invention, a method for acquiring EEG signals concurrently with application of a TMS pulse, the method including: operating an array of EEG electrodes to acquire a plurality of EEG signals concurrently with operating a TMS device to apply a TMS pulse to the head of the subject; selecting an initial set of EEG signals from the plurality of EEG signals; fitting an initial decreasing rational function to a TMS artifact component of each EEG signal of the initial set; fitting the TMS artifact component of each remaining EEG signal of the plurality of EEG signals to a linear combination of the initial decreasing rational functions; and removing the modeled TMS artifact component from each EEG signal of the plurality of EEG signals.

Furthermore, in accordance with an embodiment of the present invention, selecting the initial set includes selecting a maximum EEG signal and a minimum EEG signal.

Furthermore, in accordance with an embodiment of the present invention, the initial decreasing rational function is expressible as a quadratic polynomial in time divided by a quartic polynomial in time.

Furthermore, in accordance with an embodiment of the present invention, fitting the TMS artifact component of each remaining EEG signal includes iteratively alternating between determining values of linear combination coefficients and determining values of term coefficients.

### BRIEF DESCRIPTION OF THE DRAWINGS

In order for the present invention, to be better understood and for its practical applications to be appreciated, the following Figures are provided and referenced hereafter. It should be noted that the Figures are given as examples only and in no way limit the scope of the invention. Like components are denoted by like reference numerals.

FIG. 1A is a schematic illustration of a system for concurrent operation of electroencephalography (EEG) and transcranial magnetic stimulation (TMS), in accordance with an embodiment of the present invention.

FIG. 1B schematically illustrates an example of an interface between a skin surface and an EEG electrode of the TMS/EEG system illustrated in FIG. 1A.

FIG. 1C schematically illustrates operation of a TMS device of the TMS/EEG system illustrated in FIG. 1A.

FIG. 2A schematically illustrates a graph of an example of EEG signals that are acquired by EEG electrodes of the TMS/EEG system shown in FIG. 1A after application of a TMS pulse.

FIG. 2B schematically illustrates details of the EEG signal graph shown in FIG. 2A.

FIG. 3 is a flowchart depicting a method for performing EEG measurements concurrently with application of a TMS pulse, in accordance with an embodiment of the present invention.

FIG. 4A schematically illustrates a graph of an example of correction of EEG signals from an EEG electrode in accordance with the method shown in FIG. 3.

FIG. 4B schematically illustrates a graph of an example of correction of EEG signals from another EEG electrode.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options).

Some embodiments of the invention may include an article such as a computer or processor readable medium, or a computer or processor non-transitory storage medium, such as for example a memory, a disk drive, or a USB flash memory, encoding, including or storing instructions, computer-executable instructions, which when executed by a processor or controller, carry out methods disclosed herein.

In accordance with an embodiment of the present invention, a system is configured to enable acquisition of a plurality of electroencephalography (EEG) signals concurrently with application of transcranial magnetic stimulation (TMS). As used herein, acquisition of EEG signals concurrently with application of TMS refers to acquisition of EEG signals during a period of time in which the EEG signals are distorted by effects of the TMS pulse. In accordance with an embodiment of the present invention, meaningful (e.g., neurologically or physiologically relevant) results of the EEG measurement may be presented to an operator concurrently with their acquisition (e.g., in real time, or after an imperceptible delay).

For example, an array of EEG electrodes may be distributed across the surface of the head of a subject (e.g., across the scalp, forehead, temples, nape, face, or other parts of the head surface). Each electrode may be configured to sense electric potentials that are created by activity of the brain. One or more of the electrodes may function as a ground or reference electrode. In some cases, the electrodes may be placed at standardized or well-defined positions on the subject's head. In order to facilitate standardized or reproducible placement of the electrodes, the electrodes may be incorporated into or placed on a cap or similar device that may be worn or otherwise placed on the subject's head.

In order to facilitate sensing of the weak electric potentials that are created by activity of the brain, an electrically conducting gel or other electrically conducting material may be placed between each electrode and the skin surface of the head.

TMS may be applied by an arrangement of one or more electrically conducting coils that are placed in the vicinity of the skin of the head. The coils may be configured such that when an electrical current flows through the coils, a magnetic field is created. The coils may be configured such that maximum field strength is limited to a small region within the subject's head.

The magnetic field may typically be generated in the form of one or more short pulses. During as a result of application of the magnetic field pulse, a current is induced to flow in the brain. The induced current pulse may affect neurological function. For example, a typical length of a magnetic filed pulse may be on the order of about 100 μs.

An operator of the system (e.g., a medical practitioner or a researcher) may wish to study the effects of the induced current pulse on activity of the brain. For example, EEG signals may be analyzed to determine the effect on neurological function in the brain during the period following application of TMS. For example, in some cases, the effect neurological function may be expected to continue for a period of several hundred milliseconds up to a few seconds.

Various effects other than brain function may generate electrical potentials that may also be measured by the EEG electrodes. Such electrical potentials that are generated by effects of application of TMS other than neurological processes are referred to herein as TMS artifacts. For example, capacitances that are formed at the interface between the electrodes, the conducting gel, and the skin may generate potentials that may persist for tens or hundreds of milliseconds.

Thus the measured signal by each EEG electrode may be considered to be the sum of a neurological signal component (used herein to refer to the contribution to the measured signal by electrical potentials that are created by brain activity) and an artifact signal component (used herein to refer to the contribution to the measured signal due to interaction of the TMS field with the skin, conducting gel, electrodes, or other tissue or objects).

Application of correction techniques in accordance with embodiments of the present invention may enable extraction of the neurological signal from each measured EEG signal.

In accordance with an embodiment of the present invention, the effect of the TMS artifacts on a single EEG signal that is acquired over a period of time is modeled by a decreasing rational function of time. As used herein, an EEG signal is described as acquired by an EEG electrode if the signal represents raw data as acquired by the EEG electrode or if the signal is a result of initial processing of the raw data (e.g., any processing other than removal of an effect of a TMS artifact). Also as used herein, a decreasing rational function refers to an asymptotically decaying rational function, such that as the argument of the rational function tends to infinity, the rational function evaluated for this argument tends to zero. Thus, the effect of the TMS artifacts may be expressed as a ratio of two polynomial expressions in time where the degree of the polynomial in the denominator is greater than the degree of the polynomial in the numerator. The acquired signal may be analyzed to yield the coefficients of terms (or monomials) (referred to herein as term coefficients) of the of the numerator and denominator polynomials of the decreasing rational function. Once the term coefficients are found, the modeled artifact signal may be used to correct the measured EEG signal. For example, the decreasing rational function of time that models the artifact signal may be subtracted from the measured EEG signal to yield the neurological signal.

Typically, the decreasing rational functions that model the artifact signal components of different EEG signals will have term coefficients that differ from one another. In some cases, the term coefficients that are calculated for modeling the artifact signal components of an initial set (e.g., of one or more) of EEG signals may be utilized to shorten or to improve the accuracy of the calculation of the term coefficients of the artifact signal model for the other EEG signals that are not in the initial set. For example, the initial set of EEG signals may include a maximum EEG signal (e.g., defined as the signal that includes the maximum measured potential of all measured EEG potentials within a first predetermined time period) and a minimum EEG signal (e.g., defined as the signal that includes the minimum measured potential of all measured EEG potentials within a second predetermined time period which may be different from or identical to the first time period). As another example, the initial set may include all signals that exceed a predefined threshold within a predetermined time period, or all signals that lie below another predefined threshold during another predetermined time period.

For at least some initial sets of EEG signals, and for an artifact approximation period of time that begins following a time delay that follows application of a TMS pulse, the acquired EEG signal may be dominated by the artifact signal that is modeled by the decreasing rational function. The artifact approximation period of time may refer to a contiguous period of time, or may refer to a set of disjointed periods of time (e.g., separated from one another by gaps during which decreasing rational function approximation is not applied or is not applicable). For example, the artifact signal may be modeled by multiplicative factors or additive terms during this artifact approximation period, contributions to the artifact signal that are modeled by multiplicative factors or additive terms in addition to the contribution that is modeled by the decreasing rational function (e.g., an exponential decay). During the artifact approximation period of time, any additional multiplicative factors may be considered to be constant, and any such additive terms may be considered to be negligible.

In some cases, the decreasing rational function that models the artifact signals may be based on a model of interactions between the electrode, the skin, a conducting gel or other electrically conducting interface material, and the TMS pulse. In particular, the interfaces between the skin, conducting interface material, and the electrodes may be modeled by a two-dimensional array of leaky capacitors.

For example, the artifact signal may decay in a manner that is described in terms of a solution to a diffusion equation. This diffusion equation may incorporate, for example, the geometry of the electrodes. For example, if the electrodes are approximated by single points, a good approximation of the artifact may be expressed as

$${\sum }_{k = 1}^{K} \frac{{A}_{k}}{t + {C}_{k}} exp ( - \frac{{B}_{k}}{t + {C}_{k}} ) ,$$

where K is a positive integer, Aₖ, Bₖ, Cₖ are constants with Bₖ, Cₖ non-negative. Taylor expansion, Pade approximation or another technique may be utilized to provide a good decreasing rational function approximation of the solution of the diffusion equation for the artifacts. Also, decreasing rational functions are known to be rather well-behaved in curve-fitting procedures. Furthermore, decreasing rational functions can be constructed such that at large times, the functions asymptotically approximate a prescribed power law. Generally, once one knows the solution to a diffusion equation that describes the artifact signal, a nonlinear curve-fitting method may be applied that directly fits the solution.

In particular, in some cases, the decreasing rational function of time may consist of a quadratic polynomial in time (characterized by three term coefficients) divided by a quartic polynomial in time (characterized by five term coefficients). This decreasing rational function may be fit to a set of data points that were acquired during the artifact approximation period in order to determine the values of the coefficients. In the case of a decreasing rational function in which a quadratic polynomial is divided by a quartic polynomial, seven term coefficients are to be fit (with one of the coefficients set to a value of one).

For example, the data points to be fit may include a segment that was acquired during the artifact approximation period of an EEG signal of the initial set. The decreasing rational functions of time that are obtained by fitting decreasing rational functions to the EEG signals of the initial set are herein referred to as the initial set artifact functions. Thus, in the case of a decreasing rational function in the form of a quadratic polynomial divided by a quartic polynomial, each initial set artifact function is characterized by seven coefficients. For example, a least squares fitting technique, as is known in the art, may be applied to the data points in order to calculate the term coefficients.

The initial set artifact functions may be utilized in fitting the artifact signal components of EEG signals, herein referred to as remaining EEG signals, that were not included in the initial set, herein referred to as the remaining EEG signals. For example, the artifact signal component of each remaining EEG signal may be expressed as a decreasing rational function of time that is expressible as a linear combination of decreasing rational functions of time, each having the same functional form or structure as the initial set artifact functions (e.g., in the form of a quadratic polynomial divided by a quartic polynomial), possibly with different term coefficients. As used herein, a linear combination of functions may refer to a single function multiplied by a scaling factor, or to a sum of two or more component functions, each multiplied by a weighting factor. These linearly combined decreasing rational functions are herein referred to as component decreasing rational functions. In principle, a fitting procedure may be applied to the set of remaining EEG signals that determines the coefficients of the linear combination as well as the term coefficients of the component decreasing rational functions. However, implementing such a procedure could, in some cases, result in a fit that is not optimal. For example, the procedure of adjusting the term coefficients to yield a minimum sum of squared deviation of the fitted function from the data could result in a non-optimal fit if a local minimum of the sum is mistakenly identified to be a global minimum.

In some cases, fitting a decreasing rational function of time to a remaining EEG signal may be expedited (e.g., may enable a more rapid, accurate, or reliable calculation of the coefficients) by further constraining the form of the fitted decreasing rational function. For example, the fitted decreasing rational function of time (referred to herein as a remaining signal artifact function) may be constrained such that the component decreasing rational functions are the initial set artifact functions. In this case, each remaining signal artifact function may be expressed as a sum of the initial set artifact functions, each multiplied by a linear combination coefficient. The linear combination coefficients may be calculated by determining the values of the linear combination coefficients that enable the remaining signal artifact function to best fit the remaining EEG signal. Thus, fitting the remaining signal artifact function to a remaining EEG signal includes determining the values of the linear combination coefficients.

For example, when the initial set of EEG signals is selected to be the maximum and minimum EEG signals during the artifact approximation period (e.g., where the first and second predetermined time periods both coincide with the artifact approximation period), a linear combination of the initial set artifact functions may represent an interpolation between the maximum and minimum initial set artifact functions.

In some cases, an iterative technique may be applied to calculate the signal artifact functions. For example, as a first step of the iterative technique for generating a remaining signal artifact function, linear combination coefficients of a linear combination of the initial set artifact functions may be calculated as described above. In a second step, using the calculated linear combination coefficients, new term coefficients of the numerator and denominator polynomial functions in the component decreasing rational functions may be calculated. Iterations that include the first and second steps may be repeated until a criterion is met. For example, the criterion may include a predetermined number of iterations, or may include repeating iterations until the improvement of the fit by implementation of the latest step (e.g., as measured by goodness of fit criterion, as is known in the art) is less than a threshold value. For example, in some cases, two to three iterations were found to be sufficient.

In some cases, previously calculated term coefficients or linear combination coefficients may have been empirically found to provide an approximate fit to signals that are acquired under at least a set of measurement conditions (e.g., subjects with similarly sized heads, similarly placed electrodes, similarly applied TMS pulses, similar equipment, or other measurement conditions or criteria). In some cases, the fitting process may be expedited by utilizing these previously calculated coefficients in an initial step of the fitting process, e.g., application of an iterative fitting technique.

Once the coefficients of an artifact function, e.g., of an initial set artifact function or of a remaining signal artifact function, have been calculated, the artifact function may be utilized. For example, the artifact function may be utilized to model a contribution of the artifact signal to a measured EEG signal. The modeled artifact signal may be subtracted from, or otherwise separated from, the measured EEG signal in order to yield the neurological signal. The extracted neurological signal may be utilized to diagnose a neurological condition, or in studying brain function. Alternatively or in addition, the modeled artifact signal may utilized to study the causes of artifact signals (e.g., by comparing the modeled signal to a signal that is predicted by a model based on physical interactions) or the effects on artifact signals under various measurement conditions.

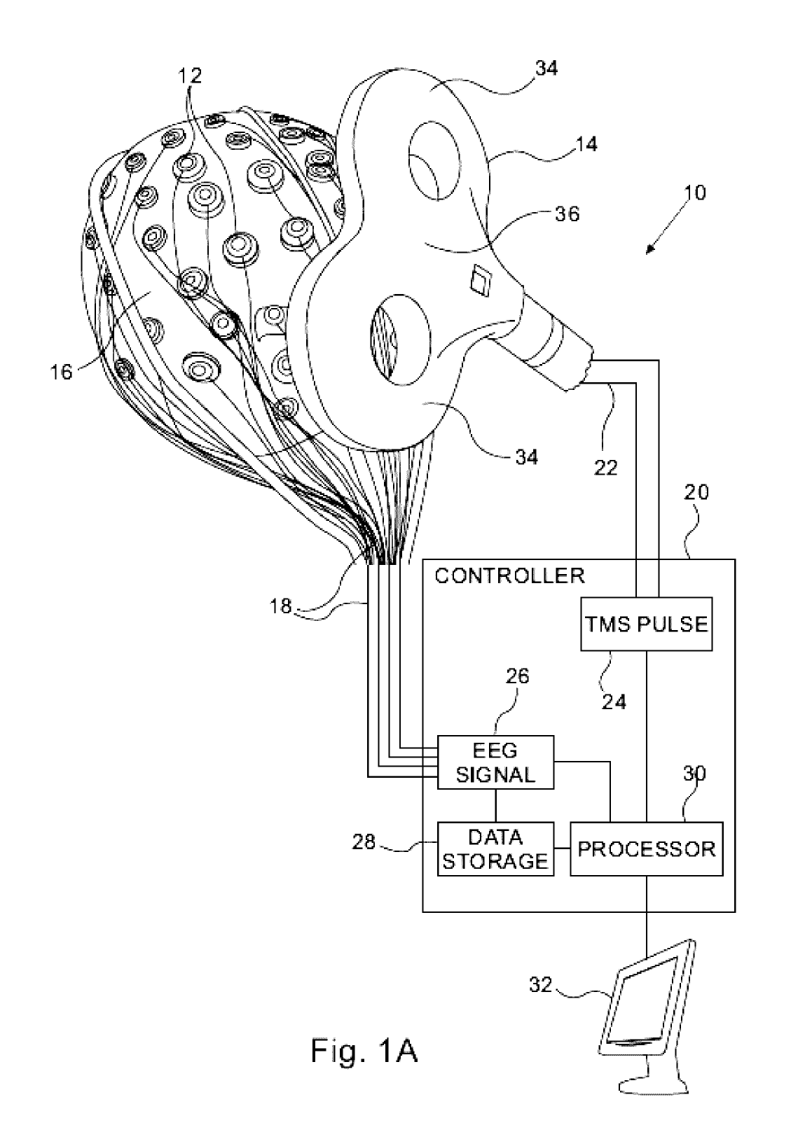

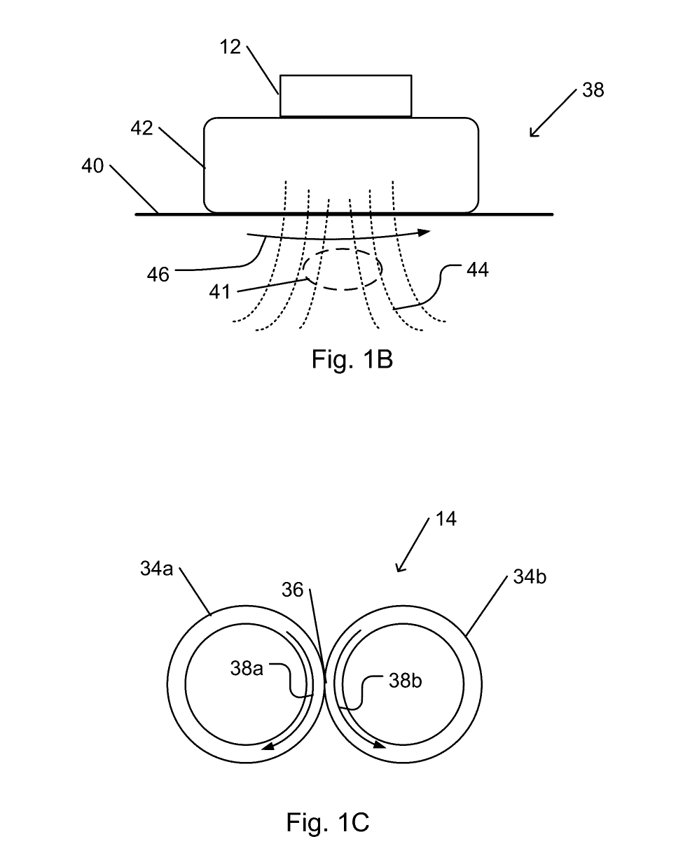

FIG. 1A is a schematic illustration of a system for EEG in the presence of TMS, in accordance with an embodiment of the present invention.

TMS/EEG system 10 includes EEG electrodes 12 and one or more TMS devices 14. EEG electrodes 12 and TMS devices 14 are configured to be operated concurrently by controller 20, e.g., in accordance with programmed instructions for operation processor 30 of controller 20.

EEG electrodes 12 are configured to be distributed over the head of a subject. For example, EEG electrodes 12 may be arranged in a two-dimensional array on the surface of an electrode cap 16. Electrode cap 16 may be configured for reproducible placement on the head of the subject. For example, electrode cap 16 is placed on the subject's head subsequent to previous placement on and removal from the subject's head. The shape, straps, openings (e.g., ear holes) may be configured such that after the subsequent placement of electrode cap 16 on the subject's head, the positions of each of EEG electrodes 12 relative to features of the subject's head its substantially identical to its positions during the previous placement. For example, EEG electrodes 12 may be configured to be arranged in a standard configuration on the subject's scalp, face, temples, nape, or other parts of the subject's head. A standard configuration may include, for example, the International 10-20 system of EEG electrode placement, or another placement scheme. In some cases, one or more of EEG electrodes 12 may be connected to ground (e.g., in some cases, an EEG electrode 12 that is placed on the subject's nape).

Typically, prior to placement of EEG electrodes 12 on the subjects head, a conducting material may be introduced into the interface between each EEG electrode 12 and the subjects head. For example, each EEG electrode 12, or part or all of the subject's head, may be coated with an electrically conducting material, e.g., an electrically conducting gel.

FIG. 1B schematically illustrates an example of an interface between a skin surface and an EEG electrode of the TMS/EEG system illustrated in FIG. 1A.

A neurological EEG signal may be generated at region 41 within the brain of a subject. In order for EEG electrode 12 to receive the neurological EEG signal, the neurological EEG signal must be conducted from region 41, via electrode-skin interface 40, to EEG electrode 12. Introduction of electrically conducting gel layer 42 into the interface between electrode-skin interface 40 and EEG electrode 12 may facilitate conduction of the neurological EEG signal (e.g., electrical currents or potentials) from region 41 interior to the subject's head, via the electrode-skin interface 40, to EEG electrode 12.

In some cases, the interfaces between adjacent layers, e.g., between electrode-skin interface 40 and electrically conducting gel layer 42, or between electrically conducting gel layer 42 and EEG electrode 12, may behave electrically as an array of leaky capacitors that is substantially parallel to electrode-skin interface 40, or as another passive electrical system. In such a case, interaction with an externally applied electromagnetic field, such as the field that is generated by a TMS pulse, may interact with electrode-skin interface 40 to generate an artifact signal that may be detected by an EEG electrode 12.

EEG electrodes 12 are connected via electrode connectors 18 to controller 20. Controller 20 includes EEG signal unit 26. EEG signal unit 26 may include circuitry that converts electrical signals that are received from EEG electrodes 12 via electrode connectors 18 to a form that is suitable for storage in data storage unit 28 and for digital processing by processor 30. For example, EEG signal unit 26 may include one or more signal amplifiers, analog-to-digital converters, electronic (analog or digital) filters, or other electronic components suitable for receiving and converting an EEG electrode signal.

Data storage unit 28 may include one or more fixed or removable, volatile or non-volatile, data storage or memory devices. Data storage unit 28 may be utilized to store one or more of programmed instructions for operation of processor 30, data or parameters for utilization by processor 30 during operation in accordance with programmed instructions, and results of operation of processor 30 in accordance with programmed instructions.

Processor 30 may include one or more processing units. For example, one or more processing units of processor 30 may be configured to operate in accordance with programmed instructions that are stored on data storage unit 28. Processor 30 may be configured to store results of operation on data storage unit 28.

Processor 30 may be configured to display or otherwise output results of operation via output device 32. Output device 32 may include a display screen, a printer, or another suitable output device. For example, output device 32 may be utilized to display one or more of a raw or processed EEG signal, parameters or values that are derived from or that characterize an EEG signal, parameters related to application of a TMS pulse or acquisition of an EEG signal, or other information.

TMS/EEG system 10 is configured to enable receiving of EEG signals from EEG electrodes 12 concurrently with operation of TMS device 14 to generate one or more TMS pulses. TMS device 14 may include one or more electrically conducting coils 34, or another arrangement of electrical conductors that is suitable or producing a magnetic field. Processor 30 may operate TMS pulse unit 24 to cause electric current to flow through electrically conducting coils 34. Typically, the electric current is in the form of one or more short direct current pulses. During operation, TMS device 14 is placed on or near the subject's head. For example, TMS device 14 may be placed at the position of a particular EEG electrode 12, or at another identifiable position relative to electrode cap 16 or to the subject's head.

FIG. 1C schematically illustrates operation of a TMS device of the TMS/EEG system illustrated in FIG. 1A.

In the configuration shown. TMS device 14 is configured such that electrical current is conducted through electrically conducting coils 34a and 34b in opposite directions. For example, current may be made to flow through electrically conducting coil 34a in the direction indicated by current flow direction 38a. Similarly, current may be made to flow through electrically conducting coil 34b in the direction indicated by current flow direction 38b. The directions of current flow directions 38a and 38b may be reversed.

In the configuration shown, current density within TMS device 14 is maximal at maximum current region 36. Thus, a magnetic field that is generated by current flow through electrically conducting coils 34a and 34b will be maximal surrounding maximum current region 36. Therefore, when TMS device 14 is placed on or near a subject's head, a maximum magnetic field is introduced in the subject's brain may have a maximum strength near maximum current region 36. For example, the maximum strength of the generated magnetic field 44 within the brain may be found approximately perpendicularly below the section of the skin surface that is nearest to maximum current region 36.

TMS device 14 is configured to be operated by TMS pulse unit 24, e.g., as controlled by processor 30 of controller 20. TMS pulse unit 24 may include circuitry (e.g., including capacitors or inductors, or other suitable circuitry) that is configured to generate large current pulses for a short period of time (e.g., lasting for approximately 100 or for another period of time), referred to herein as a TMS pulse. TMS pulse unit 24 may be configured to generate a single TMS pulse, or to generate as series of TMS pulses, e.g., periodically or otherwise at predetermined intervals.

Components of controller 20 may be housed together in a standalone device, or may be housed separately. For example, controller 20 may include one or more separately housed, or mutually remote, components. For example, one or both of EEG signal unit 26 and TMS pulse unit 24, may be connected to, or may be incorporated, into (e.g., as a circuit board), a computer that includes data storage unit 28 and processor 30. Alternatively or in addition, some of the functionality of units of controller 20 may be provided by one or more standalone devices, whiled some of the functionality is provided by components of a computer or computing system.

Operation of TMS device 14 to generate TMS pulses may, due to the changing magnetic field 44 that is generated within the brain, induce an electric current 46 to flow within a region of the brain near maximum current region 36. Induced electric current 46, generated magnetic field 44, or both, may stimulate activity of one or more regions of the brain. (It should be noted that the illustrated directions and configurations of generated magnetic field 44 and of induced electric current 46 are illustrative only, and may not correspond to actual, or physically attainable directions and configurations.) In some cases, the duration of the effect of a single TMS pulse on brain activity has been measured to be on the order of about 100 ms. The resulting activity may generate electrical activity in the brain that may be measured by EEG electrodes 12 as a neurological signal. Analysis of the neurological signal may be useful in detecting of diagnosing a defect in brain function, or in studying brain function.

Generation of a TMS pulse may induce various electrical interactions at electrode-skin surface interface 40. For example, electrical interactions may be caused by interaction of the TMS pulse with interfaces of electrode-skin interface 40 that function similarly to leaky capacitors. These electrical interactions may be measured by EEG electrodes 12 as an artifact signal. (In some cases, elimination of the artifact signal has been demonstrated by using EEG electrodes that penetrate the skin surface.)

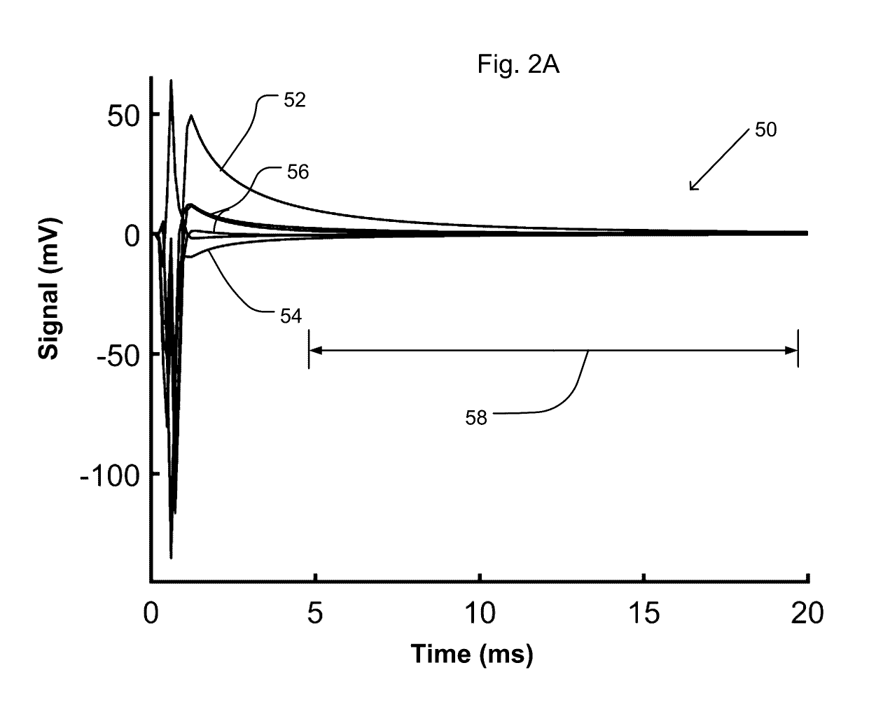

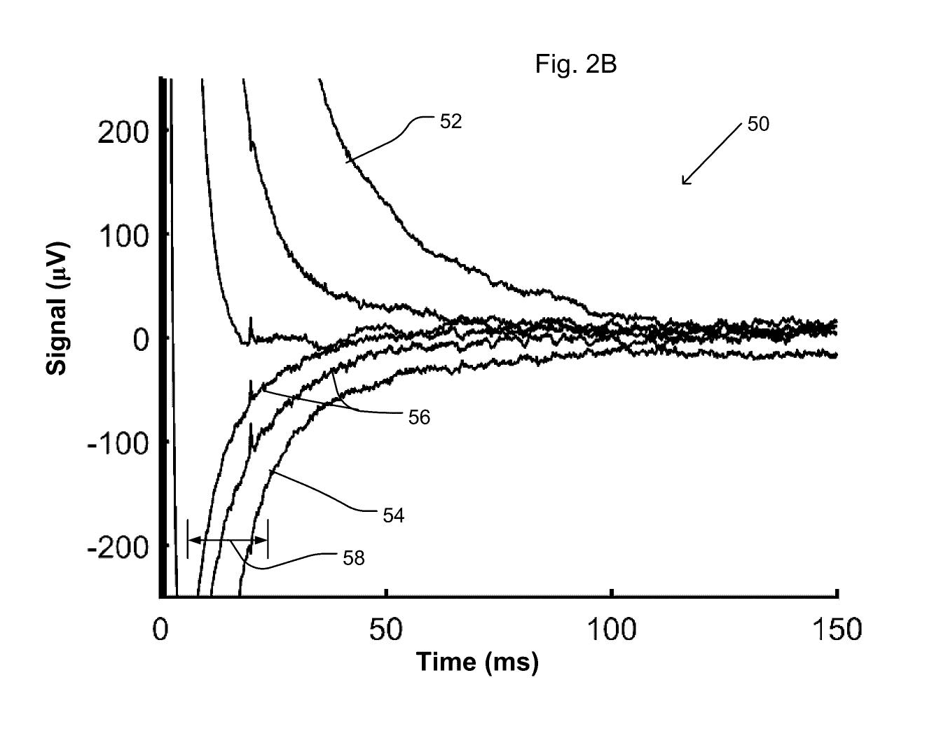

FIG. 2A schematically illustrates a graph of an example of EEG signals that are acquired by EEG electrodes of the TMS/EEG system shown in FIG. 1A after application of a TMS pulse. FIG. 2B schematically illustrates details of the EEG signal graph shown in FIG. 2A.

EEG signal graph 50 illustrates EEG signals, recorded as electrical potentials (e.g., measured in units of millivolts or microvolts) as a function of time (e.g., in units of milliseconds). In the graphs shown, each curve may represent a signal as measured, or may represent a signal after initial processing (e.g., filtering to remove interference by other nearby devices or equipment, normalizing to compensate for systematic effects, or other initial processing).

A TMS pulse, with a duration that is negligible with respect to the time scale of EEG signal graph 50 (e.g., with a duration of about 0.1 ms), was applied at time zero. As a result, each graphed EEG signal curve represents a sum of a neurological signal and an artifact signal.

In particular, maximum signal 52 includes a maximum signal value during artifact approximation period 58, and minimum signal 54 includes a minimum signal value during artifact approximation period 58. The position of artifact approximation period 58 is illustrative only, and should not be understood as representing the actual time limits of artifact approximation period 58. For example, the position of artifact approximation period 58 may depend on such factors as skin preparation and electrode geometry. In some cases, position of artifact approximation period 58 may range from about 6 ms to 8 ms after application of the TMS pulse. Remaining signals 56 include signal values that lie between the maximum and minimum signal values during artifact approximation period 58.

In general, three time periods, corresponding to differences in behavior of the EEG signal curves, such as maximum signal 52, minimum signal 54, and remaining signals 56, may be identified. Prior to artifact approximation period 58, the artifact signal is dominated by a rapidly decaying component. For example, the rapidly decaying component may correspond to an exponential decay. During artifact approximation period 58, the artifact signal may be closely approximated by a decreasing rational function. Also during artifact approximation period 58, the EEG signal is sufficiently dominated by the artifact signal so as to enable an accurate fit of the decreasing rational function to the EEG signal curve. For example, artifact approximation period 58 may be determined empirically or by considering the time span during which certain measured or calculated signals exceed a known range of physiological signals. After artifact approximation period 58, the artifact signal and the neurological signals have similar values. Therefore, although the artifact signal may still be described by the decreasing rational function, a fit to the decreasing rational function may not be accurate.

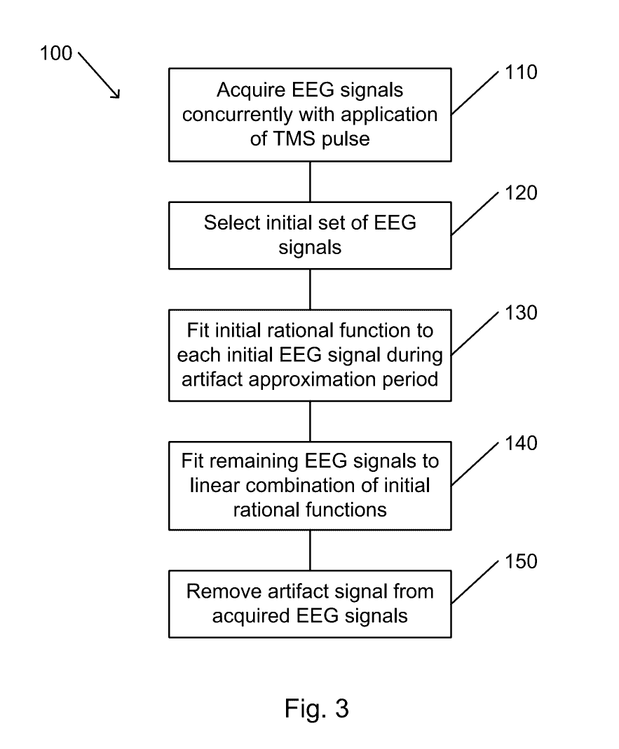

FIG. 3 is a flowchart depicting a method for performing EEG measurements concurrently with application of a TMS pulse, in accordance with an embodiment of the present invention.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

TMS/EEG method 100 may be executed by processor 30 of controller 30 of TMS/EEG system 10. For example, controller 30 may be configured to detect when EEG signals are acquired concurrently with, or immediately following, application of a TMS pulse. At that point, execution of TMS/EEG method 100 may be initiated automatically.

EEG signals are acquired concurrently with, or immediately following, application of a TMS pulse (block 110).

An initial set of one or more acquired EEG signals may be selected from among all of the acquired EEG signals (block 120). For example, an initial set of two EEG signals may be selected. In some cases, the initial set may include maximum signal 52 and minimum signal 54. The acquired EEG signals may include raw EEG signals as measured by EEG electrodes 12, or may result from preprocessed or initially processed raw EEG signals. Preprocessing or initial processing may include, for example, filtering, calibration, application of an affine-linear or nonlinear transformation, or other adjustments. Preprocessing or initial processing may include extraction or derivation of features, for example, by independent component analysis or principal component analysis such that the EEG signals represent independent components or principal components.

An artifact approximation period 58 may be identified. For example, controller 20 may include a clock or timer function that is configured to identify a time of application of a TMS pulse and to identify artifact approximation period 58.

For example, artifact approximation period 58 may be identified on the basis of an empirically identified period of time after application of a TMS pulse (e.g., about 10 ms to 30 ms, or another empirically identified period of time), or may be determined by analysis of one or more of the acquired EEG signals, e.g., by identifying time periods during which certain signals exceed the range of physiological signals.

An initial decreasing rational function f(t,P) as a function of time t and set of term coefficients P may be fit to each EEG signal of the initial set during the artifact approximation period (block 130). The decreasing rational function may be expressed as a quotient of two polynomial functions

$$f ( t , P ) = \frac{p ( t )}{q ( t )} ,$$

where

$$p ( t ) = {\sum }_{n = 0}^{N p} {p}_{n} {t}^{n} , q ( t ) = {\sum }_{n = 0}^{{N}_{q}} {q}_{n} {t}^{n} ,$$

and N_{q}>Nₚ, and P is a vector whose components consist of the term coefficients pₙ and qₙ. Thus, f(t,P) is a decreasing rational function in time. In particular, a decreasing rational function with Nₚ=2 and N_{q}=4 has been found to provide a good fit. Other polynomial quotients may be used.

When the initial set includes J EEG signals (e.g., J=2, or equal to one or more), then J initial decreasing rational functions fⱼ(t,Pⱼ) may be fit. For example, a least squares fitting technique may be applied to determine the term coefficients P that provide a best fit. Since all of the coefficients may be divided by a nonzero p or q to fix the value of one of the coefficients to 1, the fit may be used to find values of Nₚ+N_{q}−1 coefficients P.

Each of the K remaining EEG signals (outside of the initial set) may be fit to a decreasing rational function gₖ(t) that is a linear combination of the initial decreasing rational functions f(t,P) (block 140). For example,

$${g}_{k} ( t ) = {\sum }_{j ∈ J} {d}_{j , k} {f}_{j} ( t , {Q}_{j , k} ) ,$$

where d are the linear combination coefficients, and term coefficients Q may differ from initial term coefficients P.

Various techniques may be applied to expedite calculation of gₖ(t). For example, gₖ(t) may be constrained to have the form of a linear combination of the initial decreasing rational functions such that

$${g}_{k} ( t ) = {\sum }_{j ∈ J} {d}_{j , k} {f}_{j} ( t , {P}_{j} ) .$$

Each a remaining EEG signal may be fit to a linear combination of the initial decreasing rational functions to determine the linear combination coefficients d for that remaining set.

In some cases, following determination of linear combination coefficients d for a linear combination of the initial decreasing rational functions, a second step may be applied to further improve the fit. In this second step, using the previously determined linear combination coefficients d, new term coefficients Q may be fit to each remaining EEG signal. The iterative process of fitting linear combination coefficients d, followed by fitting new term coefficients Q, may be repeated a predetermined number of times. Thus, determination of fitting linear combination coefficients d is alternated with determination of new term coefficients Q. The process may be iteratively repeated until the improvement in fit (e.g., as measured by a goodness of fit criterion known in the art) falls below a predetermined threshold value, or until one or more other criteria are met. For example, 2 or 3 iterations may be sufficient in some cases.

In some cases, coefficients may have been found to be applicable to all measurements made under a set of conditions. In this case, the coefficients may be stored, e.g., on data storage unit 28, for direct application in the future (e.g., without a fitting procedure), or as an initial value for optimizing a future fitting procedure.

The fitted decreasing rational functions may be used to remove the artifact signal from the acquired EEG signals (block 150). For example, the fitted decreasing rational function may be subtracted form the EEG signal. The resulting neurological signal may be analyzed to study or diagnose the functioning of the brain.

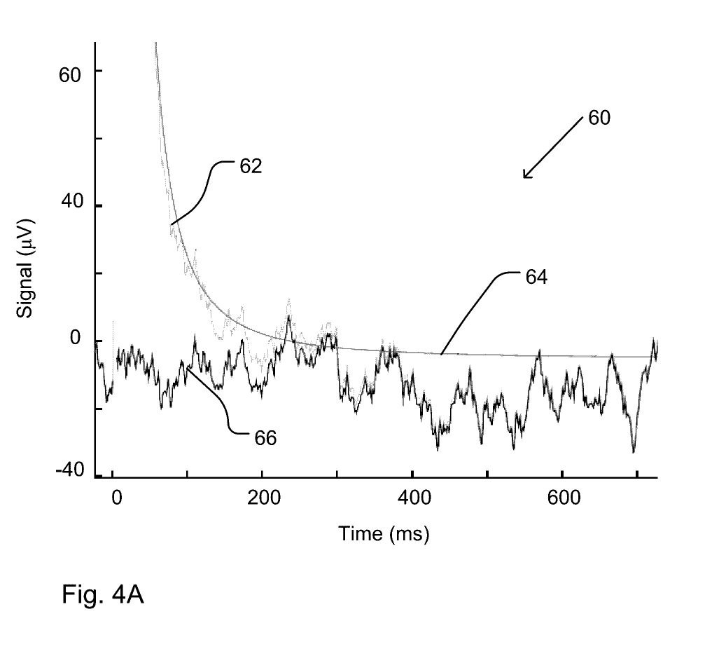

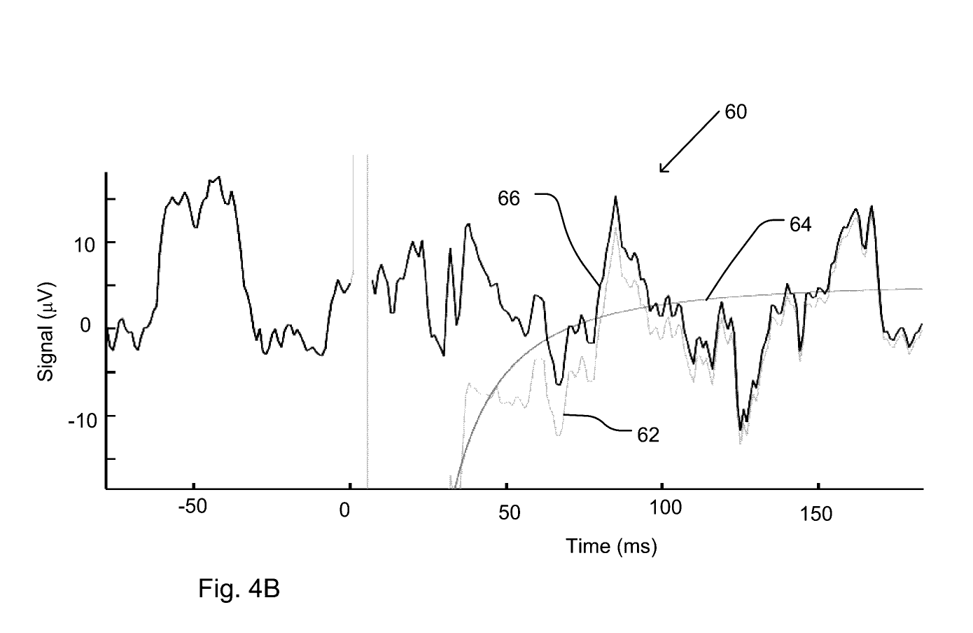

FIG. 4A schematically illustrates a graph of an example of correction of EEG signals from an EEG electrode in accordance with the method shown in FIG. 3. FIG. 4B schematically illustrates a graph of an example of correction of EEG signals from another EEG electrode.

In correction graph 60, original signal curve 62 is distorted by a large artifact signal. Decreasing rational function curve 64 was created by fitting the curve to a part of original signal curve 62 where the artifact signal dominates the neurological signal. Subtracting decreasing rational function curve 64 from original signal curve 62 yield corrected signal curve 66. Corrected signal curve 66 may be considered to illustrate the neurological signal.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A system comprising:
an array of electroencephalography (EEG) electrodes for placement on the head of a subject;
a transcranial magnetic stimulation (TMS) device; and
a controller configured to operate the array of EEG electrodes to acquire a plurality of EEG signals concurrently with operation of the TMS device to apply a TMS pulse to the head of the subject, the controller further configured to:
select an initial set of EEG signals from said plurality of EEG signals;
fit an initial decreasing rational function of time to a TMS artifact component of each EEG signal of the initial set;
fit the TMS artifact component of each remaining EEG signal of said plurality of EEG signals to a linear combination of the initial decreasing rational functions; and
correct each EEG signal of said plurality of EEG signals by removing from said each EEG signal the function that is fit to the TMS artifact component of said each EEG signal.

2. The system of claim 1, wherein the controller is configured to identify the artifact component of each EEG signal by identifying an artifact approximation period in which each EEG signal is dominated by its artifact component.

3. The system of claim 2, wherein the artifact approximation period is empirically determined.

4. The system of claim 1, wherein the controller is configured to select the initial set by selecting a maximum EEG signal and a minimum EEG signal.

5. The system of claim 1, wherein the initial decreasing rational function is expressible as a quadratic polynomial in time divided by a quartic polynomial in time.

6. The system of claim 1, wherein the controller is configured to fit linear combination to said each remaining EEG signal by determining values of linear combination coefficients.

7. The system of claim 6, wherein the controller is configured to fit the linear combination to said each remaining EEG signal by iteratively alternating between determining the values of linear combination coefficients and determining values of term coefficients.

8. The system of claim 1, wherein the controller is configured to correct each EEG signal of said plurality of EEG signals by subtracting from said each EEG signal the function that is fit to the TMS artifact component of said each EEG signal.

9. A controller of a system for acquiring EEG signals concurrently with application of TMS pulse, the controller configured to:
operate an array of EEG electrodes to acquire a plurality of EEG signals concurrently with operation of a TMS device to apply a TMS pulse to the head of the subject;
select an initial set of EEG signals from said plurality of EEG signals;
fit an initial decreasing rational function to a TMS artifact component of each EEG signal of the initial set;
fit the TMS artifact component of each remaining EEG signal of said plurality of EEG signals to a linear combination of the initial decreasing rational functions; and
remove the modeled TMS artifact component from each EEG signal of said plurality of EEG signals.

10. The controller of claim 9, configured to identify the artifact component of each EEG signal by identifying an artifact approximation period in which each EEG signal is dominated by its artifact component.

11. The controller of claim 10, wherein the artifact approximation period is empirically determined.

12. The controller of claim 9, configured to select the initial set by selecting a maximum EEG signal and a minimum EEG signal.

13. The controller of claim 9, wherein the initial decreasing rational function is expressible as a quadratic polynomial in time divided by a quartic polynomial in time.

14. The controller of claim 9, configured to fit the linear combination to said each remaining EEG signal by determining values of linear combination coefficients.

15. The controller of claim 14, configured to fit the linear combination to said each remaining EEG signal by iteratively alternating between determining the values of linear combination coefficients and determining values of term coefficients.

16. The controller of claim 9, configured to correct each EEG signal of said plurality of EEG signals by subtracting from said each EEG signal the function that is fit to the TMS artifact component of said each EEG signal.

17. A method for acquiring EEG signals concurrently with application of a TMS pulse, the method comprising:
operating an array of EEG electrodes to acquire a plurality of EEG signals concurrently with operating a TMS device to apply a TMS pulse to the head of the subject;
selecting an initial set of EEG signals from said plurality of EEG signals;
fitting an initial decreasing rational function to a TMS artifact component of each EEG signal of the initial set;
fitting the TMS artifact component of each remaining EEG signal of said plurality of EEG signals to a linear combination of the initial decreasing rational functions; and
removing the modeled TMS artifact component from each EEG signal of said plurality of EEG signals.

18. The method of claim 17, wherein selecting the initial set comprises selecting a maximum EEG signal and a minimum EEG signal.

19. The method of claim 17, wherein the initial decreasing rational function is expressible as a quadratic polynomial in time divided by a quartic polynomial in time.

20. The method of claim 17, wherein fitting the TMS artifact component of each remaining EEG signal comprises iteratively alternating between determining values of linear combination coefficients and determining values of term coefficients.
